(19) United States
(12) Patent Application Publication
(10) Pub. No.: US 2007/0148704 A1
(43) Pub. Date: Jun. 28, 2007

(54) Anti-CCPand antinuclear antibodies in diagnosis of rheumatoid arthritis

(76) Inventors: Ursula Klause, Peissenberg (DE); Hans-Peter Lehmann, Antdorf (DE); Iris Fuehling, Antdorf (DE); Susanne Schmolke, Penzberg (DE); Werner Zolg, Weilheim-Unterhausen (DE)

Correspondence Address: ROCHE DIAGNOSTICS OPERATIONS INC., 9115 Hague Road, Indianapolis, IN, 46250-0457, US

(21) Appl. No.: 11/538,198

(22) Filed: Oct. 3, 2006

Related U.S. Application Data
(60) Provisional application No. 60/724,258, filed on Oct. 6, 2005.

(51) Int. Cl. G01N 33/567; G01N 33/53

(52) U.S. Cl. 435/7.2

(57) ABSTRACT

The present invention relates to a method aiding in the assessment of rheumatoid arthritis. The method especially is used in the differential diagnosis of rheumatoid arthritis in vitro. The method is for example practiced by analyzing biochemical markers, comprising measuring in a sample both the concentration of anti-CCP and of antinuclear antibodies (ANA) correlating the concentrations determined to the diagnosis of rheumatoid arthritis. To further improve the assessment of RA in a method of this invention the level of one or more additional marker may be determined together with anti-CCP and ANA and be correlated to the absence or presence of RA. The invention also relates to the use of a marker panel comprising anti-CCP and ANA in the diagnosis of rheumatoid arthritis and it teaches a kit for performing the method of the invention.

### RELATED APPLICATIONS

This application claims priority to U.S. 60/724,258 filed Oct. 6, 2005.

### FIELD OF THE INVENTION

The present invention relates to a method aiding in the assessment of rheumatoid arthritis (RA). The method especially is used in the differential diagnosis of rheumatoid arthritis in vitro. The method is for example practiced by analyzing biochemical markers, comprising measuring in a sample both the concentration of anti-CCP (cyclic citrullinated peptides) and of antinuclear antibodies (ANA) and correlating the concentrations determined to the diagnosis of rheumatoid arthritis. To further improve the assessment of RA in a method of this invention, the level of one or more additional markers may be determined together with anti-CCP and ANA and be correlated to the absence or presence of RA. The invention also relates to the use of a marker panel comprising anti-CCP and ANA in the diagnosis of rheumatoid arthritis and it teaches a kit for performing the method of the invention.

### BACKGROUND

Rheumatoid arthritis (RA) is a chronic, inflammatory, systemic disease that produces its most prominent manifestations in affected joints, particularly those of the hands and feet. The onset of rheumatoid arthritis can occur slowly, ranging from a few weeks to a few months, or the condition can surface rapidly in an acute manner.

RA has a worldwide distribution and involves all ethnic groups. Although the disease can occur at any age, the prevalence increases with age and the peak incidence is between the fourth and sixth decade. The prevalence estimates for the North American population vary from 0.3% to 1.5%. Today over 2,500,000 individuals are diagnosed with rheumatoid arthritis in the United States alone, with some statistics indicating from 6.5 to 8 million potentially afflicted with the disease. Women are affected 2-3 times more often than men.

The early symptoms of rheumatoid arthritis are mostly joint specific such as painful joints with joint swelling or tenderness, but may also include rather non-specific manifestations like stiffness, fever, subcutaneous nodules, and fatigue. Very characteristic is the symmetric involvement of joints. The joints of the hands, feet, knees and wrists are most commonly affected, with eventual involvement of the hips, elbows and shoulders. As the disease progresses, any type of motion becomes very painful and difficult leading eventually to a loss of function of the involved joints. The more severe cases of rheumatoid arthritis can lead to intense pain and joint destruction. Some 300,000 bone and joint replacement surgical procedures are performed annually in an effort to alleviate the pain and mobility loss resultant from arthritis related joint destruction.

The most widely used system to classify RA is the American College of Rheumatology 1987 revised criteria for the classification of RA. (Arnett, F. C., et al., Arthritis Rheum. 31 (1988) 315-324). According to these criteria (known as ARA-criteria), a patient is said to have RA if the patient satisfies at least four of the following seven criteria, wherein criteria 1-4 must be present for at least six weeks: 1) morning stiffness for at least one hour, 2) arthritis of three or more joint areas, 3) arthritis of hand joints, 4) symmetrical arthritis, 5) rheumatoid nodules, 6) serum rheumatoid factor (RF), and 7) radiographic changes. These criteria have a sensitivity and specificity of approximately 90%.

The only biochemical marker generally accepted (see the above ARA-criteria) and aiding in the diagnosis of RA is the rheumatoid factor (RF) as detected in serum.

The histological changes in RA are not disease-specific but largely depend on the organ involved. The primary inflammatory joint lesion involves the synovium. The earliest changes are injury to the synovial microvasculature with occlusion of the lumen, swelling of endothelial cells, and gaps between endothelial cells, as documented by electron microscopy. This stage is usually associated with mild proliferation of the superficial lining cell layer. Two cell types constitute the synovial lining: bone marrow derived type A synoviocyte, which has macrophage features, and mesenchymal type B synoviocyte. Both cell types contribute to synovial hyperplasia, suggesting a paracrine interaction between these two cell types. This stage of inflammation is associated with congestion, oedema, and fibrin exudation. Cellular infiltration occurs in early disease and initially consists mainly of T lymphocytes. As a consequence of inflammation, the synovium becomes hypertrophic from the proliferation of blood vessels and synovial fibroblasts and from multiplication and enlargement of the synovial lining layers.

Granulation tissue extends to the cartilage and is known as pannus. The tissue actively invades and destroys the periarticular bone and cartilage at the margin between synovium and bone, known as erosive RA.

The articular manifestations of RA can be placed in two categories: reversible signs and symptoms related to inflammatory synovitis and irreversible structural damage caused by synovitis. This concept is useful not only for staging disease and determining prognosis but also for selecting medical or surgical treatment. Structural damage in the typical patient usually begins sometime between the first and second year of the disease (Van der Heijde, D. M., Br. J. Rheumatol. 34 (1995) 74-78). Although synovitis tends to follow a fluctuating pattern, structural damage progresses as a linear function of the amount of prior synovitis.

The aetiology of the early events in RA remains elusive. An autoimmune component is widely accepted today but other factors are still disputed. The possibility of a bacterial or viral infection has been vigorously pursued. All efforts to associate an infectious agent with RA by isolation, electron microscopy, or molecular biology have failed. It is possible that there is no single primary cause of RA and that different mechanisms may lead to the initial tissue injury and precipitate synovial inflammation.

Clinical signs of synovitis may be subtle and are often subjective. Warm, swollen, obviously inflamed joints are usually seen only in the most active phase of inflammatory synovitis. Cartilage loss and erosion of periarticular bone are the characteristic features of structural damage. The clinical features related to structural damage are marked by progressive deterioration functionally and anatomically. Structural damage to the joint is irreversible and additive.

The effective treatment of rheumatoid arthritis has generally comprised a combination of medication, exercise, rest and proper joint protection therapy. The therapy for a particular patient depends on the severity of the disease and the joints that are involved. Non-steroidal anti-inflammatory drugs, corticosteroids, gold salts, methotrexate and systemic immunosuppressants are widely used to reduce inflammation and joint destruction. The use of steroids and immunosuppressants, however, has significant risks and side effects both in terms of toxicity and vulnerability to potentially lethal conditions. More recently therapeutics based on “biologicals” have been introduced into RA-therapy. Such therapeutics, e.g., are soluble receptors or antibodies directed against TNF-α that significantly reduce inflammation. Though very promising, biologicals are still in limited use due to high costs.

Data from longitudinal clinical and epidemiologic studies provide guidelines for treatment. These studies emphasize 1) the need for early diagnosis, 2) identification of prognostic factors, and 3) early aggressive treatment. Earlier diagnosis and treatment, preferably within the first several months after onset of symptoms, may help prevent irreversible joint damage.

One big problem, however, is that rheumatoid arthritis and other rheumatic diseases, like for example systemic lupus erythematosus (SLE), and mixed connective tissue disease (MCTD) are not easily differentiated from one another. Tedious evaluation of disease history, clinical symptoms, X-rays and other medical examinations together with the measurement of biological markers have to be performed in order to establish a clear-cut diagnosis, e.g. of RA. In the end the physician will base its diagnosis on a combination of all the criteria he has at hand. With any biomarker or any combination of biomarkers it is always the clinician who finally establishes a diagnosis. But biomarkers are considered to be of significant help in the process of establishing e.g., the diagnosis RA.

Hence a need for methods, especially based on biochemical parameters, aiding in the assessment of rheumatoid arthritis exists. The present invention provides such methods and reagents for assessing the absence or presence of rheumatoid arthritis in vitro.

### SUMMARY OF THE INVENTION

The present invention is directed to a method for assessing rheumatoid arthritis in vitro by biochemical markers, comprising measuring in a sample the concentration of anti-CCP and antinuclear antibodies and correlating the concentrations determined to the absence or presence of rheumatoid arthritis.

The present invention also relates to the use of a marker panel comprising at least anti-CCP and antinuclear antibodies in the diagnosis of RA.

The present invention also provides a kit for performing the method according to the present invention comprising at least the reagents required to specifically measure anti-CCP and antinuclear antibodies, respectively, and optionally auxiliary reagents for performing the measurement.

In a first preferred embodiment the present invention relates to a method for assessing rheumatoid arthritis in vitro by biochemical markers, comprising measuring in a sample the concentration of anti-CCP and antinuclear antibodies and correlating the concentrations determined to the diagnosis of rheumatoid arthritis. Preferably this marker combination is used to ascertain the presence of RA.

### DETAILED DESCRIPTION OF THE INVENTION

As used herein, each of the following terms has the meaning associated with it in this section.

The articles “a” and “an” are used herein to refer to one or to more than one (i.e. to at least one) of the grammatical object of the article. By way of example, “a marker” means one marker or more than one marker.

The term “marker” or “biochemical marker” as used herein refers to a molecules to be used as a target for analyzing patient test samples. Examples of such molecular targets are proteins or polypeptides themselves as well as antibodies present in a sample. Proteins or polypeptides used as a marker in the present invention are contemplated to include any variants of said protein as well as fragments of said protein or said variant, in particular, immunologically detectable fragments. One of skill in the art would recognize that proteins which are released by cells or present in the extracellular matrix which become damaged, e.g., during inflammation could become degraded or cleaved into such fragments. Certain markers are synthesized in an inactive form, which may be subsequently activated by proteolysis. As the skilled artisan will appreciate, proteins or fragments thereof may also be present as part of a complex. Such complex also may be used as a marker in the sense of the present invention. Variants of a marker polypeptide are encoded by the same gene, but differ in their PI or MW, or both (e.g., as a result of alternative mRNA or pre-mRNA processing, e.g. alternative splicing or limited proteolysis) and in addition, or in the alternative, may arise from differential post-translational modification (e.g., glycosylation, acylation, and/or phosphorylation).

The term marker as indicated above according to the present invention also relates to antibodies present in a sample. In the case of RA these antibodies are autoantibodies, i.e. antibodies in a patient sample which bind to an antigen present in or on or produced by the patient's own cells.

The term “sample” as used herein refers to a biological sample obtained for the purpose of evaluation in vitro. In the methods of the present invention, the sample or patient sample preferably may comprise any body fluid. Preferred test samples include blood, serum, plasma, urine, saliva, and synovial fluid. Preferred samples are whole blood, serum, plasma or synovial fluid, with plasma or serum being most preferred.

As the skilled artisan will appreciate, any such diagnosis is made in vitro. The patient sample is discarded afterwards. The patient sample is merely used for the in vitro diagnostic method of the invention and the material of the patient sample is not transferred back into the patient's body. Typically, the sample is a liquid sample.

The term “assessing rheumatoid arthritis” is used to indicate that the method according to the present invention will (together with other variables, e.g., the criteria set forth by the ARA (see above)) aid the physician to establish his diagnosis of RA. As the skilled artisan will appreciate no biochemical marker is diagnostic with 100% specificity and at the same time 100% sensitivity for a given disease, rather biochemical markers are used to assess with a certain likelihood or predictive value the presence or absence of a disease. Preferably the method according to the present invention aids in assessing the presence of RA.

As the skilled artisan will appreciate, the step of correlating a marker level to the presence or absence of RA can be performed and achieved in different ways. In general a reference population is selected and a normal range established. It is no more than routine experimentation, to establish the normal range for both anti-CCP as well as antinuclear antibodies using an appropriate reference population. It is generally accepted that the normal range to a certain but limited extent depends on the reference population in which it is established. The ideal reference population is high in number, e.g., hundreds to thousands, and matched for age, gender and optionally other variables of interest. The normal range in terms of absolute values, like a concentration given, also depends on the assay employed and the standardization used in producing the assay.

The levels for anti-CCP and antinuclear antibodies can be measured and established with the assay procedures given in the examples section. It has to be understood that different assays may lead to different cut-off values.

Citrullinated peptides are antigens for rather important autoantibodies as found in the sera of patients with RA. They have been intensively studied during the past years by several groups of researchers (cf. e.g., WO 98/08946; WO 98/22503; WO 99/28344; WO 99/35167, WO 01/46222, and WO 03/050542). Recently Schellekens and co-workers (Schellekens, G. A. et al., Arthritis Rheum. 43 (2000) 155-163) reported that an ELISA-test based on specific cyclic citrullinated peptides (CCP) showed superior performance characteristics with regard to diagnostic accuracy for RA as compared to the same assay using linear peptides.

Auto-antibodies against CCP, i.e., antibodies which most likely are reactive with citrullinated polypeptides circulating in patient serum and which bind to CCP in an in vitro assay are termed “anti-CCP”. The patent application of van Venroji et al. (WO 98/22503) describes certain citrullinated peptides and shows that cyclization leads to an improved reactivity of the respective peptides. In a specific example it is shown that, if a peptide of the general formula HQCHQESTXGRSRGRCGRSGS (SEQ ID NO:1), where X stands for citrulline, is cyclisized by a disulfide bond between the two cysteine residues, the sensitivity is increased to 63% as compared to 36% to the corresponding linear peptide. As autoantibodies in patient sera having slightly different reactivity to different cyclic peptides a combination of peptides was suggested in WO 98/22503 to further improve the assay.

In a preferred embodiment anti-CCP is measured as described by van Venroij et al in WO 03/050542. In brief, a combination of peptides that contain epitope sites with the general formula X-G and X-nonG wherein X stands for citrulline, G for glycine and nonG for any of the amino acids H, I, W, S, R, K, Y, M, F, V, P, Cit or an analogue thereof is used to assess the level of anti-CCP antibodies (anti-CCP) in a sample. Specific peptides useful in such assessment are disclosed in WO 03/050542. As the skilled artisan will readily appreciate, further improvements and refinements regarding the cyclic citrullinated peptide antigen used in an assay to measure anti-CCP are possible which will e.g. result in an altered sequence of the cyclic citrullinated peptide sequence. However, such modifications will not depart from the spirit of this invention.

The antibody binding to CCP, i.e., anti-CCP, is measured in a serological assay. Preferably such assay is set up by using one or more CCP as antigen and detecting the binding of anti-CCP antibodies comprised in a sample to the CCP antigen by appropriate means.

Preferred means of detection are specific binding assays, especially immunoassays. Immunoassays are well known to the skilled artisan. Methods for carrying out such assays as well as practical applications and procedures are summarized in related textbooks. Examples of related textbooks are Tijssen, P., In: Practice and theory of enzyme immunoassays, eds. R. H. Burdon and v. P. H. Knippenberg, Elsevier, Amsterdam (1990) 221-278 and various volumes of Methods in Enzymology, eds. S. P. Colowick, N. O. Caplan and S. P., Academic Press, dealing with immunological detection methods, especially volumes 70, 73, 74, 84, 92 and 121.

Anti-CCP antibodies may be detected by homogeneous assays formats, e.g., by agglutination of latex particles coated with CCP.

Preferably a heterogeneous immunoassay is used to measure anti-CCP. Such heterogeneous measurement is based on directly or indirectly coating CCP to a solid phase, incubating the solid phase with a sample known or suspected to comprise anti-CCP antibodies under conditions allowing for binding of anti-CCP antibodies to CCP, and directly or indirectly detecting the anti-CCP antibody bound. A further assay format is the so-called double antigen bridge assay, wherein, in case of an anti-CCP measurement, CCPs are used both at the solid phase side as well as at the detection side of this immunoassay and the autoantibodies in a patient sample form a bridge between these “double” antigens. Where necessary or appropriate, washing steps are included while performing a heterogeneous immunoassay.

At the term “antinuclear antibodies” (ANA) indicate, ANA are directed against a variety of nuclear antigens and have been detected in the serum of patients with many rheumatic and non-rheumatic diseases, as well as in patients with no definable clinical syndrome. Evidence-based guidelines for the use of immunological assays detecting ANA are available (Solomon D. H., Arthritis and Rheumatism 47 (2002) 434-444).

ANA may be measured by various methods, like for example immunohistochemical methods using rodent kidney or liver cells, human epithelial-2 (HEp-2) cells, or by heterogeneous immuno assay method, like an enzyme-linked immunosorbent assay (ELISA) or a fluorescence based heterogeneous immuno assay.

In a preferred embodiment ANA is measured by an immunohistochemical method.

In most recent years significant progress has been made in the detection of ANA by heterogeneous immuno assay methods. It is now, for example, possible to include individual highly purified antigens for the various antinuclear antibodies of different specificity and to thus specifically detect the corresponding ANA. For example antibodies to double-stranded DNA, or to antigens termed SSA, SSB, Sm/RNP, centromer, etc, can now be specifically detected.

In a further preferred embodiment of the present invention the ANA-testing is performed by an heterogeneous immuno assay method. Preferably the ANA-antigens used for such testing at least include the antigens for measurement of autoantibodies to SSA60, SSA52, SSB, Jo-1, Scl70, Sm/RNP, double-stranded DNA, and centromer B peptide. In the differential diagnosis of RA a positive value for at least one of the autoantigens SSA60, SSA52, SSB, Jo-1, Scl70, Sm/RNP, double-stranded DNA, and centromer B peptide is considered as ANA-positive.

Several of the above autoantigens are described in some detail below.

SSA or Ro(SSA) Antigen

Autoantibodies to the Ro(SSA) antigen are one of the most frequent serological markers of autoimmunity in rheumatic diseases (Tan, E. M. (1989) Adv. Immunol. 44, 93; McCauliffe, D. P. & Sontheimer, R. D. (1993) J. Inv. Dermatol. 100, 73S). They are present in the serum of 50-80% of patients with Sjögren's syndrome (SS), 30-40% of patients with systemic lupus erythematosus (SLE), and 3-5% of patients with rheumatoid arthritis (RA) (Harley, J. B. et al. (1986) Arthritis Rheum. 29, 196; Reichlin, M. (1986) J. Clin. Immunol. 6, 339). Ro(SSA) antibodies target protein antigens associated with small RNA molecules known as hY-RNAs. These protein-RNA complexes are referred to as Ro-ribonucleoproteins (Ro-RNPs) and their biological function has yet to be elucidated.

Anti-Ro(SSA) positive sera may contain two different types of autoantibody; those directed to a 60 kDa and those directed to a 52 kDa polypeptide component (referred to as Ro60 and Ro52, or SSA60 and SA52, respectively) (Chan, E. K. L. & Buyon, J. P. (1994) Man. Biol. Markers Dis. (Kluwer Acad. Publ.) B4.1/1-18). While the vast majority of Ro(SSA) positive sera react with both of these components, anti-Ro60 antibody has been reported to occur without anti-Ro52 antibody only in SLE sera (Ben-Chetrit, E. et al. (1990) Arthritis Rheum. 33, 349; Slobbe, R. L. et al. (1991) Clin. Exp. Immunol. 86, 99) whereas anti-Ro52 antibody has been reported to occur in the absence of anti-Ro60 antibody in idiopathic inflammatory myopathy, dermatomyositis and scleroderma (Frank, M. B. et al. (1999) J. Autoimmun. 12, 137; Peene, I. et al. (2002) Ann. Rheum. Dis. 61, 1090). Ro52 is a member of the tripartite motif (TRIM) family of proteins. The TRIM motif includes three zinc-binding domains, a RING finger, a B-box type 1 and a B-box type 2, and a centre coiled-coil region (leucine zipper). TRIM proteins are believed to identify specific cell compartments through a process of homomultimerisation (Reymond, A. et al. (2001) EMBO J. 20, 2140).

SSB or La(SSB) Antigen

Autoantibodies to the La (SSB) antigen can be detected in the sera of up to 87% of patients with primary or secondary SS. The presence of anti-La (SSB) autoantibodies usually coincides with the presence of anti-Ro (SSA) autoantibodies, however the fact that anti-Ro autoantibodies are far more common in other rheumatological conditions such as systemic lupus erythematosis (SLE) and mixed connective tissue disease (MCTD) suggests that anti-La is more specific for primary and secondary SS than anti-Ro (8. St. Clair, E. W. (1992) Rheum. Dis. Clin. N. America 18, 359; Harley, J. B. (1989) J. Autoimmun. 2, 383). The La (SSB) antigen binds to the oligo(U) 3′termini of nascent RNA polymerase III transcripts and facilitates transcriptional termination and reinitiation by this enzyme (Stefano, J. E. (1984) Cell 36, 145; Gottlieb, E. & Steitz, J. A. (1989) EMBO J. 8, 841). It has also been reported to function as an ATP-dependent helicase able to melt RNA-DNA hybrids (Bachmann, M. et al. (1990) Cell 60, 85)

Jo1 Antigen

The most common autoantibody in Polymyosytosis and Dermatomyositosis is anti-Jo-1, occurring in 15l-20% of all myositosis patients and about 30% of adult PM patients (Nishikai, M., Reichlin, M. (1980) Arthritis Rheum. 23, 881; Targoff, I. N. (1990) Rheum. Dis. Clin. North Am. 18, 455) In 1983 the Jo-1 antigen was reported by Mathews and Bernstein (Mathews, M. B., & Bernstein, R. M. (1983) Nature 304, 177) to be histidyl-tRNA synthetase (HRS), an enzyme which catalyses the coupling of histidine to its specific tRNA before transport to the ribosome followed by incorporation into a polypeptide chain during protein synthesis.

RNP/Sm Antigens

Autoantibodies directed against the snRNP (small nuclear ribonucleoprotein) autoantigens referred to as RNP and Sm were originally detected in the sera of systemic lupus erythematosus (SLE) patients. (Tan, E. M. & Kunkel, H. G. (1966) J. Immunol 96, 464; Mattioli, M. & Reichlin, M. (1971) J. Immunol. 107, 1281). Anti-RNP antibodies were subsequently found in the sera of mixed connective tissue disease (MCTD) patients (Sharp, G. C. et al. (1972) Am. J. Med. 52, 148).

The snRNPs are a group of nuclear particles comprised of several polypeptides associated with a small nuclear RNA molecule. The most abundant snRNPs are involved in premRNA-splicing (Lührmann, R. et al. (1990) Biochim. Biophys. Acta 1087, 265).

Scl70 Antigen

Approximately 20-28% of scleroderma patients have autoantibodies to a nuclear protein referred to as Scl-70 (Douvas, A. S. et al. (1979) J. Biol. Chem. 254, 10514). In 1986 the Scl-70 antigen was identified by Shero et al. to be the superhelical DNA-relaxing enzyme topoisomerase I (Shero, J. H. et al. (1986) Science 231, 737).

Double-Stranded DNA (dsDNA)

Antibodies to double-stranded DNA are rarely seen in healthy individuals and considered a hallmark of systemic lupus erythematosus (SLE). They are usually determined by a fluorescent assay based on a DNA-containing organelle of Crithidia lucilliae, by radio immuno assay (RIA) or ELISA. It has been reported that between 50 and 90% of untreated SLE patients test positive for anti-dsDNA antibodies (Griesmacher A. and Peichl P., Clin Chem Lab Med 39 (2001) 189-208). Clinical improvements in patients with SLE are often associated with a decrease or a complete disappearance of anti-dsDNA antibodies.

Centromere B-Peptide for Detection of Anticentromere Antibodies

Anticentromere antibodies (ACA) are found in 80-90% of patients with the limited (CREST) variant of systemic sclerosis. They are directed to restricted regions of chromosomes (Barland B, and Lipstein E., Am. J. Med. 100 (1996) 16S-23S). ELISA methods based on recombinant CENP-B peptide are reported to be more sensitive in identifying ACA than immunofluorescence assays (Parveen S., et al., J. Gasteroenterol. Hepatol. 10 (1995) 438-445).

Since most of the individual autoantibodies discussed above are to a certain extend indicative for a specific subgroup of autoimmune patients, such values for one or more specific ANA will preferably be included into an evaluation algorithm in order to indicate to which sub-group of rheumatic diseases other than RA such samples will likely belong to.

In a further preferred embodiment according to the present invention the testing for anti-CCP-antibodies and the testing for ANA are performed from the same sample in one single assay procedure by employing a protein chip. In such protein chip the antigen for measurement of anti-CCP as well as the various antigens for measurement for ANA are coated onto a solid support in individual areas, the autoantibodies in the sample are bind to their corresponding autoantigen and all autoantibodies bound are detected as described in U.S. Pat. No. 6,815,217.

The ideal scenario for diagnosis would be a situation wherein a single event or process would cause the respective disease as, e.g., in infectious diseases. In all other cases correct diagnosis can be very difficult, especially when the etiology of the disease is not fully understood as is the case for RA. Therefore, generally various clinical symptoms and biological markers are considered together for diagnosis of RA. Markers can either be determined individually or in a preferred embodiment of the invention they can be measured simultaneously using a chip or a bead based array technology. The concentrations of the biomarkers are then interpreted independently using an individual cut-off for each marker or they are combined for interpretation.

In a method according to the present invention at least the concentration of the biomarkers anti-CCP and ANA, respectively, is determined and the marker combination is correlated to the absence or presence of RA, wherein in a preferred mode of correlating the measurements for anti-CCP and ANA, samples positive for anti-CCP and negative for ANA are considered indicative for RA. Samples fulfilling the requirement of testing positive for anti-CCP and negative for ANA are very likely to come from a patient with RA. Thus the mere combination of the two markers anti-CCP and ANA significantly improves the positive predictive value for RA.

This finding can be used to improve the differential diagnosis of RA. As the skilled artisan knows, it is quite difficult to differentiate between various rheumatic diseases, like e.g. SLE, MCTD and RA. Patients may have a certain amount of overlap both with regard to clinical symptoms as well as with regard to biomarkers. The present invention may aid the physician in classifying patients suspected of suffering from an autoimmune disease into one group which is likely to suffer from RA, i.e., those patients having anti-CCP but no ANA and into patients which need to undergo further diagnostic measures in order to establish a definite diagnosis, i.e. those patients testing positive for both anti-CCP and ANA.

As the skilled artisan will appreciate there are many ways to use the measurements of two or more markers in order to improve the diagnostic question under investigation. In a quite simple, but nonetheless often effective approach, a positive result is assumed if a sample is positive for at lest one of the markers investigated. This may e.g. the case when diagnosing an infectious disease, like AIDS. Frequently, however, the combination of markers is evaluated. Preferably the values measured for markers of a marker panel, e.g. for anti-CCP and ANA, are mathematically combined and the combined value is correlated to the underlying diagnostic question. Marker values may be combined by an appropriate state of the art mathematical method. Well-known mathematical methods for correlating a marker combination to a disease employ methods like, discriminant analysis (DA) (i.e. linear-, quadratic-, regularized-DA), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting/Bagging Methods), Generalized Linear Models (i.e. Logistic Regression), Principal Components based Methods (i.e. SIMCA), Generalized Additive Models, Fuzzy Logic based Methods, Neural Networks and Genetic Algorithms based Methods. The skilled artisan will have no problem in selecting an appropriate method to evaluate a marker combination of the present invention. Preferably the method used in correlating the marker combination of the invention e.g. to the absence or presence of RA is selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods), or Generalized Linear Models (i.e. Logistic Regression). Details relating to these statistical methods are found in the following references: Ruczinski, I., Kooperberg C., LeBlanc, M., Logic regression, J. of Computational and Graphical Statistics, 12 (2003) 475-511; Friedman, J. H., Regularized Discriminant Analysis, J. of the American Statistical Association, Vol. 84 (1989) 165-175; Hastie, Trevor, Tibshirani, Robert, Friedman, Jerome, The Elements of Statistical Learning, Springer Series in Statistics, 2001; Breiman, L., Friedman, J. H., Olshen, R. A., Stone, C. J. (1984) Classification and regression trees, California: Wadsworth; Breiman, L., Random Forests, Machine Learning, 45 (2001) 5-32; Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

It is preferred embodiment of the invention to use an optimized multivariate cut-off for the underlying combination of biological markers and to discriminate state A from sate B, e.g. diseased from healthy. In this type of analysis the markers are no longer independent but form a marker panel. It could be established that combining the measurements of anti-CCP and of ANA does significantly improve the diagnostic accuracy, especially the positive predictive value for RA as compared to either healthy controls or, as also assessed, as compared to patients with other rheumatic diseases. Especially the later finding is of great importance, because patients with rheumatic diseases other than RA may require quite a different mode of treatment.

Accuracy of a diagnostic method is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1—specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1—specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1—specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot. By convention, this area is always ≧0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

In a preferred embodiment the present invention relates to a method for improving the diagnostic accuracy, especially the positive predictive value, for rheumatoid arthritis versus healthy controls and/or patients suffering from other rheumatic diseases by measuring in a sample the concentration of at least anti-CCP and antinuclear antibodies and correlating the concentrations determined to the presence or absence of rheumatoid arthritis, the improvement resulting in more patients being correctly classified as suffering from RA versus healthy controls and/or patients suffering from other rheumatic diseases as compared to a classification based on anti-CCP alone. The RA marker panel comprising anti-CCP and ANA may also be used in assessing the severity of disease for patients suffering from RA.

As the skilled artisan will appreciate one or more additional biomarker may be used to further improve the assessment of RA. To illustrate this additional potential of using anti-CCP and ANA as the key markers of a panel of markers for assessment of RA the term “at least” has been used in the appending claims. With other words, the level measured for one or more additional marker may be combined with the measurement of anti-CCP and ANA in the assessment of RA.

The one or more additional marker used together with anti-CCP and ANA may be considered to be part of an RA marker panel, i.e., a series of markers appropriate to further refine the assessment of RA. The total number of markers in an RA marker panel is preferably less than 20 markers, more preferred less than 15 markers, also preferred are less than 10 markers with 8 or less markers being even more preferred. Preferred are RA marker panels comprising 3, 4, 5, or 6 markers in total.

In a preferred embodiment the present invention thus relates to a method for assessing the absence or presence of rheumatoid arthritis in vitro by biochemical markers, comprising measuring in a sample the concentration of anti-CCP, antinuclear antibodies and in addition the concentration of one or more other marker and correlating the concentrations of anti-CCP, ANA and of the one or more additional marker to the absence or presence of rheumatoid arthritis.

The measurement of anti-CCP and ANA preferably will be part of a bigger autoimmunity testing panel. Such panel testing preferably will comprise measuring anti-CCP, ANA and at least one additional marker selected from the group consisting of CRP, SAA, IL-6, S100, osteopontin, RF, MMP-1, MMP-3, hyaluronic acid, sCD14, angiogenesis markers and products of bone, cartilage or synovium metabolism.

Preferably the one or more other marker is selected from the group consisting of serum amyloid A (SAA), C-reactive protein (CRP), interleukin 6 (IL-6), S100, osteopontin, RF, matrix metalloprotease 1 (MMP-1), matrix metalloprotease 3 (MMP-3), hyaluronic acid, sCD14, angiogenesis markers and products of bone, cartilage or synovium metabolism.

Serum amyloid A (SAA) is an acute phase protein of low molecular weight of 11.7 kDa. It is predominantly synthesized by the liver in response to IL-1, IL-6 or TNF-α stimulation and is involved in the regulation of the T-cell dependent immune response. Upon acute events the concentration of SAA increases up to 1000-fold reaching one milligram per milliliter. It is used to monitor inflammation in diseases as divers as cystic fibrosis, renal graft refection, trauma or infections (Mozes, G. et al., J Trauma 29 (1980) 71-74: “Serum amyloid A: An extremely sensitive marker of tissue damage in trauma patients and indicator of acute response in various diseases”). In rheumatoid arthritis it has in certain cases been used as a substitute for CRP, but, SAA is not yet as widely accepted (Chambers R. E. et al., Ann. Rheum. Dis. 42 (1983) 665-667).

C-reactive protein (CRP) is a homopentameric Ca²⁺-binding acute phase protein with 21 kDa subunits that is involved in host defense. CRP synthesis is induced by IL-6, and indirectly by IL-1, since IL-1 can trigger the synthesis of IL-6 by Kupffer cells in the hepatic sinusoids. The normal plasma concentration of CRP is <3 μg/ml (30 nM) in 90% of the healthy population, and <10 μg/ml (100 nM) in 99% of healthy individuals. Plasma CRP concentrations can, e.g. be measured by homogeneous assay formats or ELISA. C-reactive protein is a marker for underlying systemic inflammation.

Interleukin-6 (IL-6) is a 21 kDa secreted protein that has numerous biological activities that can be divided into those involved in hematopoiesis and into those involved in the activation of the innate immune response. IL-6 is an acute-phase reactant and stimulates the synthesis of a variety of proteins, including adhesion molecules. Its major function is to mediate the acute phase production of hepatic proteins, and its synthesis is induced by the cytokines IL-1 and TNF-α IL-6 is normally produced by macrophages and T lymphocytes. The normal serum concentration of IL-6 is <5 pg/ml.

Osteopontin (OPN) is a secreted, highly acidic, calcium-binding, phosphorylated glycoprotein. Three isoforms are known that originate from alternative splicing which are either free or bound to the extracellular matrix. Through a RDG-motif of the 32 kDa-peptide backbone OPN can bind to integrins such as avβ3. Though it was originally purified from bone matrix it is expressed in numerous body fluids and tissues including milk, urine, activated T-cells, macrophages, fibroblasts, smooth muscle cells, kidney tissue and some tumor cells. Its expression is stimulated in response to several cytokines, growth factors or inflammatory mediators. Increased OPN concentrations have been associated with sepsis, metastatic cancer, cerebral ischemia, atherosclerotic plaques, granuloma formation in tuberculosis and autoimmune diseases such as multiple sclerosis (Chabas, D., et al., Science 294 (2001) 1731-1735) or RA (Petrow, P. K., et al., Arthritis Rheum. 43 (2000) 1597-1605).

Rheumatoid factors (RF) are autoantibodies directed against the constant Fe-region of immunoglobulin G molecules (Waaler, E., Acta Pathol. Microbiol. Scand. 17 (1940) 172-178; Moore, T. L., and Dorner, R. N., Clin Biochem. 26 (1993) 75-84). Though RF has some limitations it is currently the only immunologic marker of rheumatoid arthritis included in the ARA-criteria. Besides of RA it is also found in other inflammatory rheumatic diseases, non-rheumatic disease and even in healthy persons aged over 60 years (Bartfeld, H., Ann. NY Acad. Sci. 168 (1969) 30-40). RF autoantibodies belong to all immunoglobulin classes and most of the assays used today do not differentiate between the isotypes IgM, IgG and IgA. These RF-assays, also termed total-RF assays, determine mostly IgM but also cover IgG or IgA to some degree depending on the assay format and the supplier (Bas, S., et al., Ann. Rheum. Dis. 61 (2002) 505-510). More recently the RF-isotypes IgG and IgA have come into focus for the diagnosis of RA. When all three RF-isotypes are elevated the diagnostic value of the RF-assay might be improved (Swedler, W., et al., J. Rheumatol. 24 (1997) 1037-1044). Additionally some prognostic value has been ascribed to certain to these RF-isotypes. Especially, a high concentration of IgA-type RF was found to be an indicator for severe disease progression (Jorgensen, C., et al., Clin. Exp. Rheum. 14 (1996) 301-304). In a marker combination according to the present invention the marker RF can be any form of RF-determination including total RF, single specific RF-isotypes or any combination of RF-isotypes.

The family of matrix-metalloproteinases (MMPs) degrades almost all components of the extra-cellular matrix. Hence MMPs have been related to various types of cancer but also to inflammatory processes in RA, MMP-1 and MMP-3 are produced by fibroblasts, osteoblasts and endothelial cells upon stimulation by pro-inflammatory cytokines like IL-1 or TNF-α. Generally MMPs are found in the circulation as inactive pro-form and the marker MMP-1 and MMP-3, respectively, as used herein also relates to such inactive pro-form. MMP-1 and MMP-3 have been detected in synovial fluid or RA-patients and the levels are responsive to anti-TNF-α therapy. The most preferred metalloprotease to be used in an RA marker panel according to the present invention is MMP-1.

Instead of the metalloproteinases mentioned above it is also possible to used their corresponding inhibitors collectively referred to as tissue inhibitors of matrix metalloproteinases (TIMPs), eg. MMP-1 and MMP-3 are in vivo inactivated by TIMP-1 a sialoglycoprotein of 29.5 kD that forms a 1:1 stoichiometric complex with the MMPs. The relation of TIMP-1 and TIMP-2 to the destruction of cartilage has been investigated in RA (Ishiguro, N., et al., Arthritis Rheum. 44 (2001) 2503-2511).

S100-proteins form a constantly increasing family of Ca²⁺-binding proteins that today includes more than 20 members. The physiologically relevant structure of S100-proteins is a homodimer but some can also form heterodimers with each other, e.g. S100A8 and S100A9. The intracellular functions range from regulation of protein phosphorylation, of enzyme activities, or of the dynamics of the cytoskeleton to involvement in cell proliferation and differentiation. As some S100-proteins are also released from cells, extracellular functions have been described as well, e.g., neuronal survival, astrocyte proliferation, induction of apoptosis and regulation of inflammatory processes. S100A8, S100A9, the heterodimer S100A8/A9 and S100A12 have been found in inflammation with S100A8 responding to chronic inflammation, while S100A9, S100A8/A9 and S100A12 are increased in acute inflammation. S100A8, S100A9, S100A8/A9 and S100A12 have been linked to different diseases with inflammatory components including some cancers, renal allocraft rejection, colitis and most importantly to RA (Burmeister, G., and Gallacchi, G., Inflammopharmacology 3 (1995) 221-230; Foell, D., et al., Rheumathology 42 (2003) 1383-1389). The preferred S100 markers for use in an RA marker panel according to the present invention are S100A8, S100A9, S100A8/A9 heterodimer and S100A12.

CD14 is a membrane protein of pro-monocytes, monocytes, macrophages, and activated granulocytes where it serves as a receptor for lipopolysaccharide. It induces the secretion of cytotoxic and immunomodulating factors like reactive oxygen (O₂), tumor necrosis factor (TNF-α), interleukins (IL-1, IL-6 and IL-8) and platelet-activating factor (PAF). Membrane bound CD14 is shed to give soluble CD14 (sCD14) in response to activating or differentiating factors such as IFNγ or TNFα. The physiological function of sCD14 is not yet entirely clear. Since inflammatory and immune processes are involved in RA and other autoimmune diseases, sCD14 was also investigated in such diseases. When anti-CD14 therapy was evaluated as a new therapeutic option in RA previously elevated concentrations of sCD14 rapidly decreased and synovitis was reduced (Horneff, G., et al., Clin. Exp. Immunol. 91 (1993) 207-213).

The glycosaminoglycan hyaluronic acid is one of the macromolecules essential for the function of a joint. It is synthesized by fibroblasts and other specialized connective tissue cells. Hyaluronic acid is involved in formation of the extracellular matrix and in cell to cell contacts. High concentrations are found in synovial fluid where it is responsible for the retention of water thereby contributing to the lubrication of joints. In RA the synthesis of hyaluronic acid is stimulated by the proinflammatory mediators IL-1 and TNF-α leading to increased serum/plasma levels (Sawai, T., and Uzuki, M., Connective Tissue 33 (2001) 253-259).

A feature of rheumatoid arthritis is the invasion of joints with proliferating synovial tissue also known as pannus. A significant part of the pannus consists of blood vessels that supplies nutrients to the growing tissue. Therefore, molecules relevant in angiogenesis have been investigated in RA also, both as RA markers but also as therapeutic targets (Brenchley, P. E. C., Clin. Exp. Immunol. 121 (2000) 426-429). Amongst these the vascular endothelial growth factor (VEGF) has been evaluated in more detail. VEGF is a secreted glycoprotein that is spliced to four different isoforms. Two of these isoforms are readily diffusible while the remaining isoforms bind tightly to heparin and are mostly found in association with heparin containing proteoglycans. VEGF acts as a chemokine on endothelial cells, monocytes and osteoblasts ultimately leading to neovascularization and increased microvascular permeability. VEGF has been detected in synovial fluid and serum of RA patients (Lee, S. S., et al., Clin. Exp. Rheumathology 19 (2001) 321-324; Ballara, S., Arthritis Rheum. 44 (2001) 2055-2064). Preferably, the marker of angiogenesis is VEGF.

The most prominent joint tissues are bone, cartilage and the synovium. Since rheumatoid arthritis is a destructive disease these tissues will be most affected. They are a likely source of potential biological markers in the field of RA. In principle these markers may come not only from the destruction of the respective tissue but also from a deregulated and/or ineffective repair process. The experienced artisan will understand that markers of bone, cartilage or synovium metabolism can originate either from synthesis or from destruction of these tissues. The various markers of bone, cartilage and/or synovium metabolism can be delineated from two different groups of proteins. They come either from the numerous types of collagen or from non-collagenous proteins. Non-collagenous proteins are often involved in the formation of the extracellular matrix. Some of these markers can be found in all three tissues in varying amounts.

Markers and products of bone and/or cartilage metabolism include both markers of bone and/or cartilage degradation as well as markers of bone and/or cartilage formation. Preferred markers derived from collagen metabolism are markers such as:
Pyridinoline (PYD), deoxy-pyridinoline (DPD) and Glc-Gal-PYD: Pyridinoline (PYD) stabilizes collagen by cross-linking the strands of the collagen triple helix. The chemical structure of PYD is very stable and can be found in serum and urine as an end product of collagen degradation (Knott, L., and Bailey, A. J., Bone 22 (1998) 181-187). It has been linked to arthritis (Kaufmann, J., et al., Rheumatology 42 (2003) 314-320). PYD monitors cartilage involvement of joint destruction since it is released from cartilage and only to some degree from bone while its close cousin deoxy-pyridinoline (DPD) originates mostly from bone. All three markers have been linked to arthritis (Kaufmann, supra). The glycosylated form Glc-Gal-PYD has mostly been found in synovial tissue (Gineyts, E., et al., Rheumatology 40 (2001) 315-323).
Cross-linked telopeptides: CTX-I, CTX-II, NTX-I and the LQ-epitope which are cross-linked telopeptides either from the C- or N-terminus of collagens type I or type II, respectively, and of which β-CTX-I is also known as β-CrossLaps® (Bonde, M., et al., Clin. Chem. 40 (1994) 2022-2025). Type I collagen carboxyterminal telopeptide (ICTP) refers to a fragment and marker of type I collagen which originally has been derived from type I collagen by cyanobromide cleavage.
Linear peptides derived from collagen: The assay termed Cartilaps® measures a linear peptide that is derived from the C-terminal region of collagen type II.
Modified amino acids: Collagen comprises modified amino acids like hydroxyproline and galactosyl hydroxylysine which may be used as a marker of collagen break-down (Al-Dehaimi, A. W., et al., Clin. Chem. 45 (1999) 676-681).
Collagen neoepitopes: Col2-3/4 and CIIN are neoepitopes generated by the initial cleavage of collagen II by collagenases (Billinghurst, R. C. et al., J. Clin. Invest. 99 (1997) 1534-1545).
Collagen markers considered reflecting bone formation: The N-terminal as well as the C-terminal pro-peptide of type I collagen (PINP and PICP), respectively, are clipped from the precursor polypeptide (procollagen) during/after synthesis and considered markers of bone formation. PIICP is the corresponding pro-peptide from collagen type II, whereas PIIINP is derived from collagen III.

Preferably the marker of bone and/or cartilage metabolism also may be a non-collagenous marker, like: CS846, which is a chondriotin sulfate epitope created during aggrecan synthesis; cartilage oligomeric matrix protein (COMP) that has bridging functions in cartilage (Saxne, T., and Heinegard, D., Br. J. Rheumatol. 31 (1992) 583-591); cartilage intermediate layer protein (CILP), which is a matrix protein of cartilage (Lorenzo, P., et al., J. Biol. Chem. 273 (1998) 23463-23468); cartilage matrix proteins 1-3 also known as matrilins; chondromodulins that act as signaling molecules in cartilage (Suzuki, F., Connect. Tissue Res. 35 (1996) 303-307); cartilage derived retinoic acid-sensitive protein (CD-RAP) or MIA, which has a yet to be defined function in chondrocyte modulation (Müller-Ladner, U., et al., Rheumatology 38 (1999) 148-154); osteocalcin, which is synthesized by osteoblasts, belongs to the major non-collagen matrix protein of bone and is used to monitor bone turnover (Gundberg, C. M., et al., J. Clin. Ligand Assay 21 (1998) 128-138); and the bone sialoproteins, which are major non-collagen matrix proteins of bone, such as bone sialoprotein II, now known as bone sialoprotein, which e.g., has been evaluated as marker for bone turn-over (Saxne, T., et al., Arthritis Rheum. 38 (1995) 82-90).

Products of metabolism within the synovium which may be used as a marker in assessing RA include: CTX-III, which is a telopeptide derived from collagen type III, YKL40 the later being a chitinase 3 like protein of the extracellular matrix (Johansen, J. S., et al., Scand. J. Rheumatol. 30 (2001) 297-304), and aggrecan, which is a building block of proteoglycans as well as its degradation product keratan sulfate.

Preferably the RA marker panel comprises at least three markers, wherein anti-CCP, ANA and a third marker selected from the group consisting of CRP, IL-6, S100, osteopontin RF, MMP-1, MMP-3, hyaluronic acid, and a product of collagen metabolism are contained.

In the assessment of RA a marker panel comprising anti-CCP, ANA and S100, especially, S100A12 is preferred.

A further preferred panel of RA markers comprises anti-CCP, ANA and hyaluronic acid.

As mentioned further above (see ARA criteria)—despite severe limitations—the rheumatoid factor (RF) currently is the only biochemical marker generally accepted to aid in establishing the diagnosis of RA. It is clearly expected that the marker combination of the present invention will significantly improve the diagnosis of RA and will supplement or might be even finally replace the RF assay. The use of a marker panel comprising at least anti-CCP and ANA in the diagnosis of RA therefore represents a further preferred embodiment of the present invention.

As the skilled artisan will appreciate one or more additional marker may be used to further improve the diagnostic accuracy, or, where required increase the diagnostic sensitivity at the expense of specificity or vice versa. In some diagnostic areas, e.g., in the detection of an HIV-infection sensitivity is of utmost importance. The high sensitivity required may be achieved at the expense of specificity, leading to an increased number of false positive cases. In other cases, e.g. as a simple example, when assessing blood group antigens, specificity is of paramount importance.

A further preferred embodiment relates to the use of a marker panel in the diagnosis of RA the panel comprising anti-CCP, antinuclear antibodies and at least one additional marker selected from the group consisting of SSA, CRP, IL-6, S100, osteopontin, RF, MMP-1, MMP-3, hyaluronic acid, sCD14, angiogenesis markers and products of bone, cartilage or synovium metabolism.

The method of the present invention may also be of help in monitoring the course of disease. This may be achieved by measuring in a patient sample anti-CCP and ANA as well as optionally additional markers at various points in time and comparing the absolute and/or the relative levels of the markers at these different time points. It thus is further preferred to use the method according to the present invention to monitor the course of disease in a patient with RA.

It is also recognized that the present invention may be of help in assessing the efficacy of any treatment for RA. The efficacy of treatment will be reflected by changes in the marker level. If a treatment has the desired effect at least one of the two marker levels of anti-CCP or ANA will decrease. The method according to the present invention thus preferably is also used to assess the efficacy of treatment. The same phenomenon, i.e. a reduction in marker level of at least one of anti-CCP or ANA can easily be applied for selection of the right drug as well as the most appropriate dosing of drugs in RA. The use of a method of this invention in selection of the right drug and/or the most appropriate dosing is also preferred.

The method of the present invention may also enable the selection and identification of new drugs in the field of RA. This application represents a further preferred embodiment.

It will also be a great advantage that sub-groups of patients can now be identified for and in clinical studies which differ in their level of anti-CCP and ANA and to correlate this difference in marker level to the efficacy of the drug under investigation.

The present invention also relates to a kit for performing the method of this invention comprising the reagents required to specifically measure anti-CCP and antinuclear antibodies, respectively. The kit may optionally comprise auxiliary reagents for performing the measurement of both anti-CCP and ANA.

The following examples are provided to aid in the understanding of the present invention, the true scope of which is set forth in the appended claims. The full disclosure of the documents cited is included by reference. It is understood that modifications can be made in the procedures set forth without departing from the spirit of the invention.

### SPECIFIC EMBODIMENTS

### Example 1

Immunological Multiparametric Chip Technique IMPACT—General Procedure

A black polystyrene chip with a surface area of about 2.5×6 mm is coated completely with a streptavidin layer. Onto this streptavidin surface we apply lines of identical reagent spots (about 20 spots per line and reagent) using ink-jet technology. Each spot is about 150 μm in diameter and contains biotinylated binding reagents capable of specifically binding with an analyte (e.g., an antigen or an antibody) in a sample.

Each sample is diluted 1:10 using sample dilution buffer and 40 μl of diluted sample is applied per chip and incubated. The assay is performed on an automated pre-prototype instrument.

The diluted sample is incubated for 6 min at 37° C. During this incubation each analyte contained in the sample binds to its specific binding reagent. The sample is then aspirated and the chip is washed using washing buffer. Afterwards the chip is incubated for 3 min at 37° C. with a antibody-conjugate, which is digoxigenylated and which specifically binds to the analyte bound via the biotinylated binding reagent to the spots. After a further washing step the chip is incubated with a antibody-conjugate to digoxin, which conjugate is labeled with a fluorescent label, for 3 min at 37° C. After a further washing step and aspiration of surplus reagent the fluorescence label is excited, light is detected by a CCD-camera and light intensity is transformed into analyte concentrations.

### Example 2

Specific Assays for Measurement of Anti-CCP and of ANA

For measurement of anti-CCP the CCP-peptides as disclosed in WO 03/050542 are biotinylated and used.

For measurement of ANA individual biotinylated antigens are used, i.e., native ANA antigens such as SSA60, SSA52, SSB, Jo-1, Scl70, RNP, Sm; double-stranded DNA, centromer B peptide. A sample is recorded as positive for ANA if it test positive for at least one of these autoantigens.

The sample is diluted 1:10 in sample dilution buffer and 40 μl of diluted sample are incubated for 6 min at 37° C. Autoantibodies out of the sample bind to their specific antigens. The sample is aspirated and the chip is washed using washing buffer. Afterwards the chip is incubated for 3 min at 37° C. with an antibody-conjugate which is digoxigenylated and which specifically binds the human IgG antibodies out of the sample that are bound to the antigens located in the spots. After a further washing step the chip is incubated with a antibody-conjugate to digoxin, which conjugate is labeled with a fluorescent label, for 3 min at 37° C. After a further washing step and aspiration of surplus reagent the fluorescence label is excited, light is detected by a CCD-camera and light intensity is transformed into analyte concentrations.

Sample Dilution Buffer

50 mM Tris, pH 7.6,
150 mM NaCl,
0.1% detergent (Polydocanol),
0.6% bovine serum albumin (BSA),
0.2% preservative (Oxypyrion+Methylisothiazol-Hydrocloride (MIT) 1:1)
   Washing Buffer
10 mM Tris pH 8.2,
0.01% Polydocanol,
0.001% Oxypyrion,
0.001% MIT
   Results

Depending on the group of patients investigated, the percentage of samples positive for anti-CCP as well as the percentage of patients positive for both anti-CCP as well as ANA varies. The positive predictive value (PPV) for RA can be improved for that sub-group of patients having a positive anti-CCP and a negative ANA as compared to the PPV for all patients testing positive for anti-CCP irrespective of their ANA-status.

What is claimed is:

1. A method for diagnosing rheumatoid arthritis in a patient comprising
measuring in a sample from said patient a concentration of anti-CCP (cyclic citrullinated peptides) antibodies and a concentration of antinuclear antibodies (ANA), and
correlating the concentrations measured to a diagnosis of rheumatoid arthritis.

2. The method of claim 1, further comprising identifying the sample as being positive for anti-CCP and negative for ANA.

3. The method of claim 1 wherein said diagnosis is a differential diagnosis for rheumatoid arthritis.

4. The method according to claim 1, further comprising measuring the concentration of a marker selected from the group consisting of C-reactive protein, serum amyloid A, interleukin 6, S100 proteins, osteopontin, rheumatoid factor, matrix metalloprotease 1, matrix metalloprotease 3, hyaluronic acid, sCD14, angiogenesis markers, and products of bone, cartilage, or synovium metabolism.

5. A kit for performing the method of claim 1 comprising reagents for measuring anti-CCP and antinuclear antibodies.
